(19) United States
(12) Patent Application Publication
(10) Pub. No.: US 2016/0213800 A1
(43) Pub. Date: Jul. 28, 2016

(54) Scent preventer

(71) Applicant: Jeffrey L. Tedesco, Manahawkin, NJ (US)

(72) Inventor: Jeffrey L. Tedesco, Manahawkin, NJ (US)

(21) Appl. No.: 14/544,583

(22) Filed: Jan. 26, 2015

(51) Int. Cl. A61L 9/01; A01M 99/00; C01D 7/00; A01M 31/00

(52) U.S. Cl. CPC A61L 9/01; A01M 31/00; A01M 99/00; C01D 7/00

(57) ABSTRACT

A scent preventer comprising sodium bicarbonate and methods of its use are disclosed comprising an odorless plastic bag into which, prior to going into the field, the clothes of a hunter are inserted together with the scent preventer of a measured amount of sodium bicarbonate (Na HCO3). Then the bag is closed, shaked, and stored for up to two days prior to removal of the clothes of the hunter which are thereby rendered odor and scent free.

### BACKGROUND OF THE INVENTION

Every experienced hunter knows that game animals have a very highly developed sense of smell. Such game animals as deer. elk, moose, etc. are able to detect the windborne scent of a hunter, his clothes and equipment from a far distance, which results in the game animals running away and out of range from the area of the hunter. Therefore, the hunter must, often with difficulty and strenuous exertion, circle the game so that he is downwind.from the animal in order that it can not detect his odor or scent.

There are over 12.5 million hunters, in the United States alone, who spend over 22 billion dollars per year in an attempt to prevent equipment scent, cover scent, scent eliminators and lures. With nearly 12.5 million hunters in the United States, spending nearly 220 million days in the woods, fields, and wetlands each year, they are individually spending nearly $1,990.00 per year on prior art and ineffective scent preventers.

### OBJECTS OF THE INVENTION

The objects of the present invention are to:
1. provide an effective and economical scent preventer for use by hunters of game animals; and
2. provide simple methods for using the scent preventer.

### BRIEF DESCRIPTION OF THE DRAWINGS

FIG. 1. Illustrates a side perspective view of the scent preventer of the present invention, and

FIG. 2. Illustrates a side sectional view of the method of use of the scent preventer.

### DETAILED DESCRIPTION

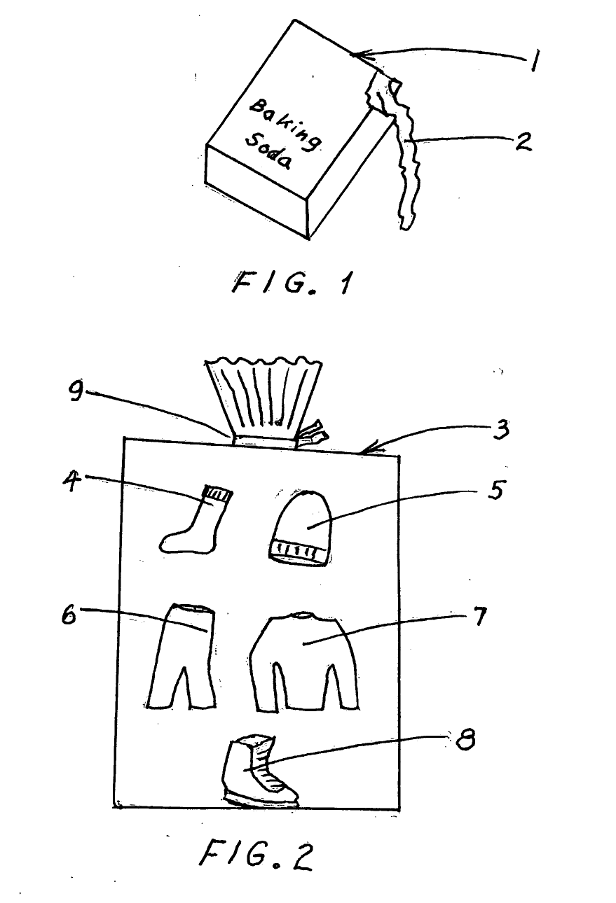

FIG. 1 illustrates a package 1 of the scent preventer 2 of the present invention, which is sodium bicarbonate commonly known as baking soda with the chemical formula.of NaHCO3. The use of baking soda powder 2 alone as a scent preventer is disclosed as a safe, economical and effective use for eliminatimg the scent of hunters and, their clothes, such as hats, gloves, socks, boots, etc of the hunter in the field.

FIG. 2 illustrates the method of using the scent preventer 2 by first laying out the hunting clothes and boots, generally shown as 4, 5, 6, 7 and 8, on the floor or ground. (rubber boots and clothes do not need to be treated with the scent protector 2. An amount of the scent protector 2, about 32 ounces, is then sprinkled over the hunting clothes and boots, 4. 5. 6. 7 and 8, which are then put into an odorless plastic container, a plastic bag 3 of approximately 40 gallon size. The plastic bag 3 is then closed and tied off at the top with a wire closure 9. The plastic bag 3 is then shaked for about 30 seconds. When ready to go hunting, the hunter opens the plastic bag 3 and puts on the clothing and boots 4, 5, 6, 7 and, 8 which are now odor and scent free so that the hunter can go into the field without having his presence detected in any way by the game. Alternatively, the hunter can utilize an odorless plastic storage box and lid for the plastic bag 3 and wire closure 9. It must be appreciated that the hunting clothes and boots, generally shown as 4,5,6,7 and 8 can also include other items such as masks, scarves, jackets, coats, parkas, blankets, underwear, etc. The varying mount of all such items can be treated by proportionally increasing or decreasing the amount of the scent protector 2 and the size of the plastic container.

In addition to using the scent protector on the hunter,s clothes, the hunter can also use the scent protector 2 as a mouthwash and as a bodywash to eliminate his personal scent. It will be understood by those skilled in the art that the present invention may be embodied in other specific forms. Without departing from the scope of the invention disclosed and that the embodiments disclosed herein are in all respects illustrative and no restrictive. Those skilled in the art of the present invention will recognize that other embodiments using the concepts described herein are also possible.

What is claimed are:

1. A scent protecter comprising sodium bicarbonate powder for use by a hunter before going into the field so as to prevent the sensing of the hunter by the game being hunted.

2. A method for using sodium bicarbonate powder as a scent protecter for hunters comprising:
sprinkling the sodium bicarbonate powder onto the clothes of the hunter,
placing the sprinkled clothes into an odorless plastic container.
closing the plastic container,
shaking the closed plastic container; and
removing the clothes from the plastic container for wearing by the hunter before the hunter goes into the field so as to prevent the odor and scent of the hunter not being sensed by the game. being hunted.
